SEQUENCE LISTING (2 sequences)

SEQ ID NO: 1 - DNA, 21 bp, Homo sapiens
aaaguugucauggaugacctt

SEQ ID NO: 2 - DNA, 21 bp, Homo sapiens
ggucauccaugacaacuuutt

(19) United States
(12) Patent Application Publication
(10) Pub. No.: US 2008/0124730 A1
(43) Pub. Date: May 29, 2008

(54) Modified siRNA Constructs for Detecting RISCs

(75) Inventors: Kai Qin Lao, Pleasanton, CA (US); Neil A. Straus, Emeryville, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 11/870,384

(22) Filed: Oct. 10, 2007

Related U.S. Application Data
(60) Provisional application No. 60/828,927, filed on Oct. 10, 2006.

(51) Int. Cl. C12Q 1/68; C07H 21/04

(52) U.S. Cl. 435/6; 536/22.1

(57) ABSTRACT

The present teachings provide novel methods, compositions, and kits for detecting siRNA-containing RISCs. In some embodiments, modified siRNA constructs are employed that contain an anti-sense strand and a sense strand, wherein the anti-sense strand comprises a 3′ end, wherein the 3′ end comprises a fluorophore, and wherein the sense strand comprises a 5′ end, wherein the 5′ end comprises a quencher. Following transfection, uptake of the anti-sense strand by RISC liberates the fluorescent signal, allowing for detection of siRNA-containing RISCs.

### CROSS REFERENCE TO RELATED APPLICATIONS

This application claims a priority benefit under 35 U.S.C. § 119(e) to U.S. Application No. 60/828,927, filed Oct. 10, 2006, the entire contents of which are incorporated herein by reference.

### FIELD

The present teachings relate generally to molecular biology, and in particular to methods, compositions, and kits for detecting the formation of siRNA-containing RISCs.

### INTRODUCTION

RNAi is increasing accepted as a potentially powerful clinical tool to silence genes (Nobel committee, 2006). For example, siRNAs are one type of molecule that can be used to reduce the expression of those genes that are causative components of disease. This approach has naturally arisen from the use of siRNAs as effective analytical tools in gene expression studies. Many of the rules of siRNA strand selection, siRNA incorporation into RISC and siRNA function within RISC, have now been worked out. For example, it is known that the basic double stranded siRNA rule for efficient strand incorporation is that a 21-23 nucleotide incorporated strand must have a 5′ phosphate end (PO4) paired with a complimentary strand so that there is a three prime overhang. It is also known that there is an asymmetry to the incorporation, with 5′ PO4 ends of lower stability being preferentially incorporated in the RISC structure (Schwarz et al., Cell, 115:199, (2003), Khvorova et al., Cell 115:209, (2003)). Furthermore, in situations where both the sense and anti-sense strands are incorporated into RISCs, the incorporation of sense strands can lead to undesirable off-target effects (Jackson et al., Nat. Biotech. 21:635, (2003)).

The scientific literature not only describes the molecular characteristics of RNAi from siRNA, but has also demonstrated some of the necessary requirements for incorporation of siRNA into RISC. Both the general characteristics and necessary conditions of RNAi have been investigated. For example, Martinez et al., showed that the 5′PO4 and the 3′ overhang was absolutely required for the RISC incorporated strand, but RNAi by this strand seemed independent of blocking agents on the other strand ends (Cell 110:563, (2002)). Therefore according to this work, siRNA could be constructed with moieties on three other ends of siRNA, and RNAi can still occur.

### SUMMARY

In some embodiments, the present teachings provide a method for detecting the formation of an siRNA-containing RISC comprising; providing a modified siRNA construct, wherein the modified siRNA construct comprises an anti-sense strand and a sense strand, wherein the anti-sense strand comprises a 3′ end, wherein the 3′ end comprises a fluorophore, and wherein the sense strand comprises a 5′ end, wherein the 5′ end comprises a quencher; transfecting the modified siRNA construct into a sample; and, measuring fluorescence to detect the formation of the siRNA-containing RISC.

In some embodiments, the present teachings provide a method for detecting the formation of an siRNA-containing RISC comprising; providing a modified siRNA construct, wherein the modified siRNA construct comprises an anti-sense strand and a sense strand, wherein the anti-sense strand comprises a 3′ end, wherein the 3′ end comprises a quencher, and wherein the sense strand comprises a 5′ end, wherein the 5′ end comprises a fluorophore; transfecting the modified siRNA construct into a sample; and, measuring fluorescence; to detect the formation of the siRNA-containing RISC.

In some embodiments, the present teachings provide a composition comprising; a modified siRNA construct, wherein the modified siRNA construct comprises an anti-sense strand and a sense strand, wherein the anti-sense strand comprises a 3′ end, wherein the 3′ end comprises a fluorophore, and wherein the sense strand comprises a 5′ end, wherein the 5′ end comprises a quencher.

In some embodiments, the present teachings provide a composition comprising; a modified siRNA construct, wherein the modified siRNA construct comprises an anti-sense strand and a sense strand, wherein the anti-sense strand comprises a 3′ end, wherein the 3′ end comprises a quencher, and wherein the sense strand comprises a 5′ end, wherein the 5′ end comprises a fluorophore.

In some embodiments, the present teachings provide a composition comprising a RISC and an anti-sense strand of a modified siRNA construct, wherein the anti-sense strand comprises a 3′ end, wherein the 3′ end comprises a fluorophore.

In some embodiments, the present teachings provide a composition comprising a RISC and an anti-sense strand of a modified siRNA construct, wherein the anti-sense strand comprises a 3′ end, wherein the 3′ end comprises a quencher.

In some embodiments, the present teachings provide a kit for detecting the formation of an siRNA-containing RISC, comprising; a modified siRNA construct, wherein the modified siRNA construct comprises an anti-sense strand and a sense strand, wherein the anti-sense strand comprises a 3′ end, wherein the 3′ end comprises a fluorophore, and wherein the sense strand comprises a 5′ end, wherein the 5′ end comprises a quencher; and, transfection reagents.

In some embodiments, the present teachings provide a kit for detecting the formation of an siRNA-containing RISC, comprising; a modified siRNA construct, wherein the modified siRNA construct comprises an anti-sense strand and a sense strand, wherein the anti-sense strand comprises a 3′ end, wherein the 3′ end comprises a quencher, and wherein the sense strand comprises a 5′ end, wherein the 5′ end comprises a fluorophore; and, transfection reagents.

### DRAWINGS

FIG. 1 depicts an illustrative schematic according to some embodiments of the present teachings.

FIG. 2 depicts an illustrative schematic according to some embodiments of the present teachings.

### DESCRIPTION OF EXEMPLARY EMBODIMENTS

It is to be understood that both the foregoing general description and the following detailed description are exemplary and explanatory only and are not intended to limit the scope of the current teachings. In this application, the use of the singular includes the plural unless specifically stated otherwise. Also, the use of “comprise”, “contain”, and “include”, or modifications of those root words, for example but not limited to, “comprises”, “contained”, and “including”, are not intended to be limiting. The term and/or means that the terms before and after can be taken together or separately. For illustration purposes, but not as a limitation, “X and/or Y” can mean “X” or “Y” or “X and Y”.

The section headings used herein are for organizational purposes only and are not to be construed as limiting the described subject matter in any way. All literature and similar materials cited in this application, including, patents, patent applications, articles, books, treatises, and internet web pages are expressly incorporated by reference in their entirety for any purpose. In the event that one or more of the incorporated literature and similar defines or uses a term in such a way that it contradicts that term's definition in this application, this application controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### SOME DEFINITIONS

As used herein, the term “modified siRNA construct” refers to a complex containing a sense strand and an anti-sense strand. The sense strand is 21-23 nucleotides in length and has a 5′ end containing a moiety such as a fluorophore or a quencher, and the anti-sense stand is 21-23 nucleotides in length and has a 3′ end containing a moiety such as a fluorophore or a quencher.

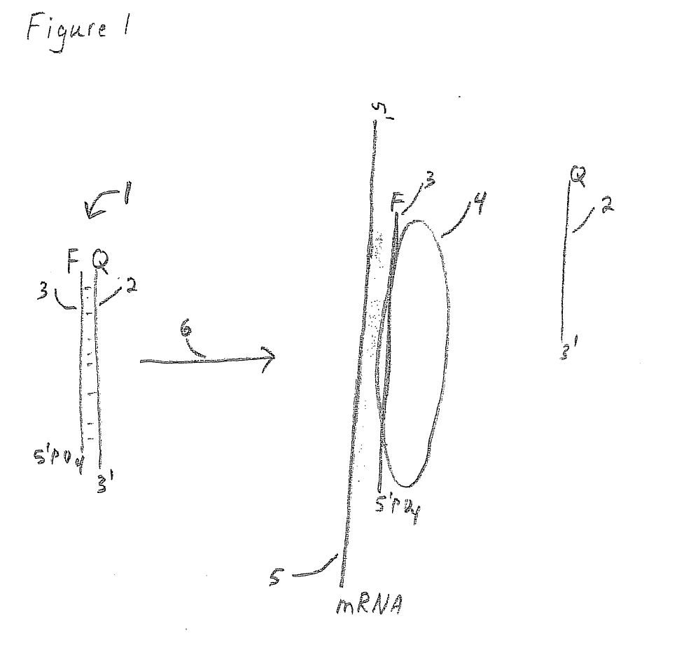

In some embodiments, the present teachings provide a method of detecting the formation of a siRNA-containing RISC. One illustrative embodiment is depicted in FIG. 1. Here, a modified siRNA construct (1) is shown containing a sense strand (2) and an anti-sense strand (3). The sense strand has a 5′ end containing a quencher (Q), and the anti-sense stand has a 3′ end containing a fluorophore (F). Following transfection (6), the anti-sense strand (3) of the modified siRNA complex can interact with RISC (4). Without being limited to any mechanistic theory, the 5′ end of the sense strand is hypothesized to be prevented from entering a RISC (4) by the presence of the quencher (Q). However, the anti-sense strand can enter the RISC due to its 5′ PO4 group, thereby allowing for detection due to separation of the anti-sense strand's fluorophore (FAM) from the sense strand's quencher (Q). The RISC complex can degrade a corresponding mRNA (5).

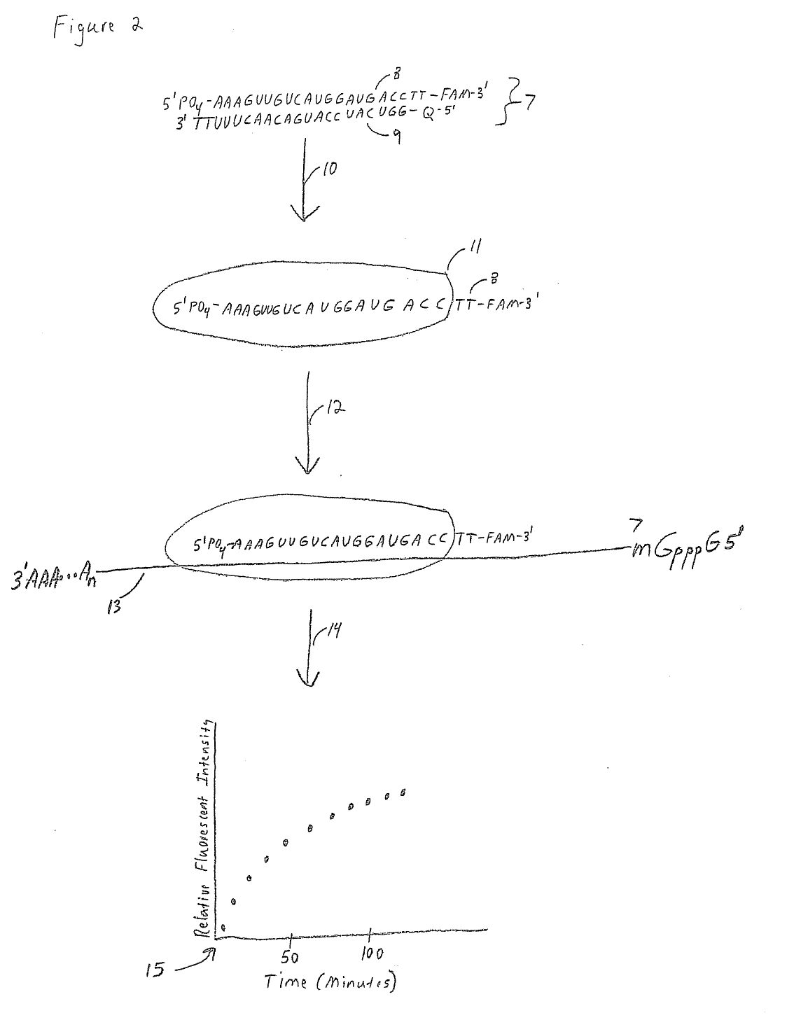

FIG. 2 depicts another illustrative embodiment according to the present teachings. Here, a modified siRNA construct (7) is shown containing a sense strand (9) and an anti-sense strand (8). The anti-sense strand has a 3′ end containing a fluorophore (F), and the sense stand has a 5′ end containing a quencher (Q). Following transfection (10), the anti-sense strand (8) of the modified siRNA complex can interact with RISC (11). Without being limited to any mechanistic theory, the 5′ end of the sense strand is hypothesized to be prevented from entering the RISC (11) by the presence of the quencher (Q). However, the anti-sense strand can enter the RISC due to its 5′ PO4 group, thereby allowing for detection due to separation of the anti-sense strand's fluorophore from the sense strand's quencher. Degradation (12) of a corresponding mRNA (13) can occur by RISC. Fluorescent measurements (14) can be taken at any time throughout this process. For example, a graphing can be performed to show the relevant fluorescent intensity as a function of time, as depicted in (15).

Thus, in some embodiments, the present teachings provide a method for detecting the formation of an siRNA-containing RISC comprising; providing a modified siRNA construct, wherein the modified siRNA construct comprises an anti-sense strand and a sense strand, wherein the anti-sense strand comprises a 3′ end, wherein the 3′ end comprises a fluorophore, and wherein the sense strand comprises a 5′ end, wherein the 5′ end comprises a quencher; transfecting the siRNA construct into a sample; and, measuring fluorescence to detecting the formation of the siRNA-containing RISC. Such detection can provide for the subcellular localization of the siRNA-containing RISC by the fluorescence imparted by the anti-sense strand.

Of course, the 3′ end of the anti-sense can contain a quencher rather than a fluorophore, such that in some embodiments the present teachings provide a method for detecting the formation of an siRNA-containing RISC comprising; providing a modified siRNA construct, wherein the modified siRNA construct comprises an anti-sense strand and a sense strand, wherein the anti-sense strand comprises a 3′ end, wherein the 3′ end comprises a quencher, and wherein the sense strand comprises a 5′ end, wherein the 5′ end comprises a fluorophore; transfecting the modified siRNA construct into a sample; and, measuring fluorescence to detect the formation of the siRNA-containing RISC.

In some embodiments, the 3′ end of the sense strand further comprises a cell uptake element. In some embodiments, the cell uptake element is an HIV tat transduction domain. In some embodiments, the cell uptake element is a G-PNA.

The present teachings also provide for novel compositions. For example, in some embodiments, the present teachings provide a composition comprising a RISC and an anti-sense strand of a modified siRNA construct, wherein the anti-sense strand comprises a 3′ end, wherein the 3′ end comprises a fluorophore. In some embodiments, the present teachings comprises a composition comprising a RISC and an anti-sense strand of a modified siRNA construct, wherein the anti-sense strand comprises a 3′ end, wherein the 3′ end comprises a quencher.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y. all of which are herein incorporated in their entirety by reference for all purposes.

In some embodiments, the sense strand of the modified siRNA construct is 21-23 nucleotides in length. In some embodiments, the anti-sense stand of the modified siRNA construct is 21-23 nucleotides in length. Methods for preparing suitable sense and anti-sense strands containing fluorophore/quencher pairs can be found for example in Livak et al. (PCR Methods Appl. 1995 June; 4(6):357-62) and U.S. Pat. No. 5,876,930 to Livak et al.). Quenchers are also available from various commercial sources, such as Epoch Biosciences. Additional illustrative constructs useful in the present teachings can be found in U.S. patent application Ser. Nos. 11/291,444 and 11/172,280. Additional strategies for generating modified siRNA constructs that contain PNA can be found in U.S. patent application Ser. No. 11/166,031 to Zon. Further, the sense strand and the anti-sense strand of the modified siRNA constructs of the present teachings can employ nucleotides as well as nucleotide analogs, including synthetic analogs having modified nucleoside base moieties, modified sugar moieties, and/or modified phosphate groups and phosphate ester moieties. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C₁-C₆ alkyl or C₅-C₁₄ aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT published application nos. WO 98/22489, WO 98/39352, and WO 99/14226). Exemplary LNA sugar analogs within a polynucleotide include, but are not limited to, the structures:

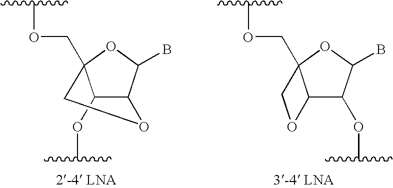

where B is any nucleotide base.

Modifications at the 2′- or 3′-position of ribose include, but are not limited to, hydrogen, hydroxy, methoxy, ethoxy, allyloxy, isopropoxy, butoxy, isobutoxy, methoxyethyl, alkoxy, phenoxy, azido, amino, alkylamino, fluoro, chloro and bromo. Nucleotides include, but are not limited to, the natural D optical isomer, as well as the L optical isomer forms (see, e.g., Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleotide base is purine, e.g. A or G, the ribose sugar is attached to the N⁹-position of the nucleotide base. When the nucleotide base is pyrimidine, e.g. C, T or U, the pentose sugar is attached to the N¹-position of the nucleotide base, except for pseudouridines, in which the pentose sugar is attached to the C5 position of the uracil nucleotide base (see, e.g., Kornberg and Baker, (1992) DNA Replication, 2^{nd} Ed., Freeman, San Francisco, Calif.).

One or more of the pentose carbons of a nucleotide may be substituted with a phosphate ester having the formula:

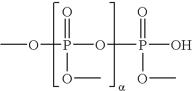

where α is an integer from 0 to 4. In certain embodiments, α is 2 and the phosphate ester is attached to the 3′- or 5′-carbon of the pentose. In certain embodiments, the nucleotides are those in which the nucleotide base is a purine, a 7-deazapurine, a pyrimidine, or an analog thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group may include sulfur substitutions for the various oxygens, e.g. α-thio-nucleotide 5′-triphosphates. For a review of nucleotide chemistry, see: Shabarova, Z. and Bogdanov, A. Advanced Organic Chemistry of Nucleic Acids, VCH, New York, 1994.

The fluorophores of the present teachings comprise a resonance-delocalized system or aromatic ring system that absorbs light at a first wavelength and emits fluorescent light at a second wavelength in response to the absorption event. A wide variety of such dye molecules are known in the art, and can be employed in the present teachings. For example, fluorescent dyes can be selected from any of a variety of classes of fluorescent compounds, such as xanthenes, rhodamines, fluoresceins, cyanines, phthalocyanines, squaraines, and bodipy dyes. In some embodiments, the dye comprises a xanthene-type dye, which contains a fused three-ring system of the form:

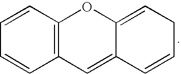

This parent xanthene ring may be unsubstituted (i.e., all substituents are H) or can be substituted with one or more of a variety of the same or different substituents, such as described below. In some embodiments, the dye contains a parent xanthene ring having the general structure:

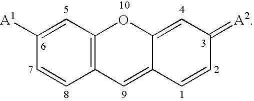

In the parent xanthene ring depicted above, A¹ is OH or NH₂ and A² is O or NH₂⁺. When A¹ is OH and A² is O, the parent xanthene ring is a fluorescein-type xanthene ring. When A¹ is NH₂ and A² is NH₂⁺, the parent xanthene ring is a rhodamine-type xanthene ring. When A¹ is NH₂ and A² is O, the parent xanthene ring is a rhodol-type xanthene ring. In the parent xanthene ring depicted above, one or both nitrogens of A¹ and A² (when present) and/or one or more of the carbon atoms at positions C1, C2, C4, C5, C7, C8 and C9 can be independently substituted with a wide variety of the same or different substituents. In some embodiments, typical substituents can include, but are not limited to, —X, —R, —OR, —SR, —NRR, perhalo (C₁-C₆) alkyl, —CX₃, —CF₃, —CN, —OCN, —SCN, —NCO, —NCS, —NO, —NO₂, —N₃, —S(O)₂O⁻, —S(O)₂OH, —S(O)₂R, —C(O)R, —C(O)X, —C(S)R, —C(S)X, —C(O)OR, —C(O)O⁻, —C(S)OR, —C(O)SR, —C(S)SR, —C(O)NRR, —C(S)NRR and —C(NR)NRR, where each X is independently a halogen (preferably —F or Cl) and each R is independently hydrogen, (C₁-C₆) alkyl, (C₁-C₆) alkanyl, (C₁-C₆) alkenyl, (C₁-C₆) alkynyl, (C₅-C₂₀) aryl, (C₆-C₂₆) arylalkyl, (C₅-C₂₀) arylaryl, heteroaryl, 6-26 membered heteroarylalkyl 5-20 membered heteroaryl-heteroaryl, carboxyl, acetyl, sulfonyl, sulfinyl, sulfone, phosphate, or phosphonate. Moreover, the C1 and C2 substituents and/or the C7 and C8 substituents can be taken together to form substituted or unsubstituted buta[1,3]dieno or (C₅-C₂₀) aryleno bridges. Generally, substituents that do not tend to quench the fluorescence of the parent xanthene ring are preferred, but in some embodiments quenching substituents may be desirable. Substituents that tend to quench fluorescence of parent xanthene rings are electron-withdrawing groups, such as —NO₂, —Br, and —I. In some embodiments, C9 is unsubstituted. In some embodiments, C9 is substituted with a phenyl group. In some embodiments, C9 is substituted with a substituent other than phenyl. When A¹ is NH₂ and/or A² is NH₂⁺, these nitrogens can be included in one or more bridges involving the same nitrogen atom or adjacent carbon atoms, e.g., (C₁-C₁₂) alkyldiyl, (C₁-C₁₂) alkyleno, 2-12 membered heteroalkyldiyl and/or 2-12 membered heteroalkyleno bridges. Any of the substituents on carbons C1, C2, C4, C5, C7, C8, C9 and/or nitrogen atoms at C3 and/or C6 (when present) can be further substituted with one or more of the same or different substituents, which are typically selected from —X, —R′, ═O, —OR′, —SR′, ═S, —NR′R′, ═NR′, —CX₃, —CN, —OCN, —SCN, —NCO, —NCS, —NO, —NO₂, ═N₂, —N₃, —NHOH, —S(O)₂O—, —S(O)₂OH, —S(O)₂R′, —P(O)(O⁻)₂, —P(O)(OH)₂, —C(O)R′, —C(O)X, —C(S)R′, —C(S)X, —C(O)OR′, —C(O)O⁻, —C(S)OR′, —C(O)SR′, —C(S)SR′, —C(O)NR′R′, —C(S)NR′R′ and —C(NR)NR′R′, where each X is independently a halogen (preferably —F or —Cl) and each R′ is independently hydrogen, (C₁-C₆) alkyl, 2-6 membered heteroalkyl, (C₅-C₁₄) aryl or heteroaryl, carboxyl, acetyl, sulfonyl, sulfinyl, sulfone, phosphate, or phosphonate.

Exemplary parent xanthene rings include, but are not limited to, rhodamine-type parent xanthene rings and fluorescein-type parent xanthene rings.

In one embodiment, the dye contains a rhodamine-type xanthene dye that includes the following ring system:

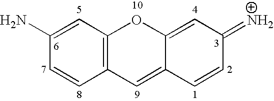

In the rhodamine-type xanthene ring depicted above, one or both nitrogens and/or one or more of the carbons at positions C1, C2, C4, C5, C7 or C8 can be independently substituted with a wide variety of the same or different substituents, as described above for the parent xanthene rings, for example. C9 may be substituted with hydrogen or other substituent, such as an orthocarboxyphenyl or ortho(sulfonic acid)phenyl group. Exemplary rhodamine-type xanthene dyes can include, but are not limited to, the xanthene rings of the rhodamine dyes described in U.S. Pat. Nos. 5,936,087, 5,750,409, 5,366,860, 5,231,191, 5,840,999, 5,847,162, and 6,080,852 (Lee et al.), PCT Publications WO 97/36960 and WO 99/27020, Sauer et al., J. Fluorescence 5(3):247-261 (1995), Arden-Jacob, Neue Lanwellige Xanthen-Farbstoffe für Fluoreszenzsonden und Farbstoff Laser, Verlag Shaker, Germany (1993), and Lee et al., Nucl. Acids Res. 20:2471-2483 (1992). Also included within the definition of “rhodamine-type xanthene ring” are the extended-conjugation xanthene rings of the extended rhodamine dyes described in U.S. application Ser. No. 09/325,243 filed Jun. 3, 1999.

In some embodiments, the dye comprises a fluorescein-type parent xanthene ring having the structure:

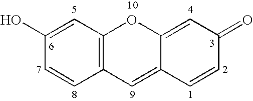

In the fluorescein-type parent xanthene ring depicted above, one or more of the carbons at positions C1, C2, C4, C5, C7, C8 and C9 can be independently substituted with a wide variety of the same or different substituents, as described above for the parent xanthene rings. C9 may be substituted with hydrogen or other substituent, such as an orthocarboxyphenyl or ortho(sulfonic acid)phenyl group. Exemplary fluorescein-type parent xanthene rings include, but are not limited to, the xanthene rings of the fluorescein dyes described in U.S. Pat. Nos. 4,439,356, 4,481,136, 4,933,471 (Lee), 5,066,580 (Lee), 5,188,934, 5,654,442, and 5,840,999, WO 99/16832, and EP 050684. Also included within the definition of “fluorescein-type parent xanthene ring” are the extended xanthene rings of the fluorescein dyes described in U.S. Pat. Nos. 5,750,409 and 5,066,580. In some embodiments, the dye comprises a rhodamine dye, which can comprise a rhodamine-type xanthene ring in which the C9 carbon atom is substituted with an orthocarboxy phenyl substituent (pendent phenyl group). Such compounds are also referred to herein as orthocarboxyfluoresceins. In some embodiments, a subset of rhodamine dyes are 4,7,-dichlororhodamines. Typical rhodamine dyes can include, but are not limited to, rhodamine B, 5-carboxyrhodamine, rhodamine X (ROX), 4,7-dichlororhodamine X (dROX), rhodamine 6G (R6G), 4,7-dichlororhodamine 6G, rhodamine 110 (R110), 4,7-dichlororhodamine 110 (dR110), tetramethyl rhodamine (TAMRA) and 4,7-dichloro-tetramethylrhodamine (dTAMRA). Additional rhodamine dyes can be found, for example, in U.S. Pat. Nos. 5,366,860 (Bergot et al.), 5,847,162 (Lee et al.), 6,017,712 (Lee et al.), 6,025,505 (Lee et al.), 6,080,852 (Lee et al.), 5,936,087 (Benson et al.), 6,111,116 (Benson et al.), 6,051,719 (Benson et al.), 5,750,409, 5,366,860, 5,231,191, 5,840,999, and 5,847,162, U.S. Pat. No. 6,248,884 (Lam et al.), PCT Publications WO 97/36960 and WO 99/27020, Sauer et al., 1995, J. Fluorescence 5(3):247-261, Arden-Jacob, 1993, Neue Lanwellige Xanthen-Farbstoffe für Fluoresenzsonden und Farbstoff Laser, Verlag Shaker, Germany, and Lee et al., Nucl. Acids Res. 20(10):2471-2483 (1992), Lee et al., Nucl. Acids Res. 25:2816-2822 (1997), and Rosenblum et al., Nucl. Acids Res. 25:4500-4504 (1997), for example. In some embodiments, the dye comprises a 4,7-dichloro-orthocarboxyrhodamine. In some embodiments, the dye comprises a fluorescein dye, which comprises a fluorescein-type xanthene ring in which the C9 carbon atom is substituted with an orthocarboxy phenyl substituent (pendent phenyl group). One typical subset of fluorescein-type dyes are 4,7,-dichlorofluoresceins. Typical fluorescein dyes can include, but are not limited to, 5-carboxyfluorescein (5-FAM), 6-carboxyfluorescein (6-FAM). Additional typical fluorescein dyes can be found, for example, in U.S. Pat. Nos. 5,750,409, 5,066,580, 4,439,356, 4,481,136, 4,933,471 (Lee), 5,066,580 (Lee), 5,188,934 (Menchen et al.), 5,654,442 (Menchen et al.), 6,008,379 (Benson et al.), and 5,840,999, PCT publication WO 99/16832, and EPO Publication 050684. In some embodiments, the dye comprises a 4,7-dichloro-orthocarboxyfluorescein. In some embodiments, the dye can be a cyanine, phthalocyanine, squaraine, or bodipy dye, such as described in the following references and references cited therein: U.S. Pat. Nos. 5,863,727 (Lee et al.), 5,800,996 (Lee et al.), 5,945,526 (Lee et al.), 6,080,868 (Lee et al.), 5,436,134 (Haugland et al.), U.S. Pat. Nos. 5,863,753 (Haugland et al.), 6,005,113 (Wu et al.), and WO 96/04405 (Glazer et al.)

Detection of the siRNA-containing RISCs can employ any of a variety of fluorescence detection systems. For example, FMAT-based detection can be employed using the 8200 Cellular Detection System, commercially available from Applied Biosystems. Such detection can occur in real-time, or as an end-point read.

In some embodiments, the modified siRNA constructs can be co-transfected into cells along with a labeled oligonucleotide that is complementary to a messenger RNA (mRNA). Such a mRNA probe can be labeled with a distinct fluorophore. Visualization can be accomplished using two channels, one channel for the fluorophore in the modified siRNA construct, and one channel for the fluorophore in the mRNA probe. Using such an approach can allow for co-localization of a mRNA with its corresponding RISC.

The modified siRNA constructs, and mRNA probes if present, can be introduced into cells using any of a variety of molecular biology techniques, including transfection using CaCl2, various liposome-mediated approaches, as well as cell uptake elements such as the HIV tat transduction domain (Nagahara et al., Nature Medicine 4:1449, 1998), and G-PNAs (Zhou et al., JACS 125:6878, 2003). A variety of commercial sources for transfection reagents exist, including FuGENE® from Applied Science, in vivo-jetPEI® from Polyplus Transfection, Express-si Delivery Kit from Panomics, HiPerFect Transfection Reagent from Qiagen, SatisFection™ from Stratagene, and Lipofectamine™ RNAiMAX Transfection Reagent from Invitrogen, and the Silencer® Transfection Kits from Ambion. For additional transfection approaches of siRNAs, see Gilmore et al., Curr Drug Deliv (2006) April: 3(2): 147-5, and Cheng et al., Nucleic Acids Research 2005 33(4):1290-1297. The modified siRNA constructs of the present teachings can also be used to perform in situ hybridization. Illustrative in situ hybridization procedures can be found in the product literature for the mRNA Locator™ In Situ Hybridization Kit commercially available from Ambion.

### Certain Exemplary Kits

The instant teachings also provide kits designed to expedite performing certain of the disclosed methods. Kits may serve to expedite the performance of certain disclosed methods by assembling two or more components required for carrying out the methods. In certain embodiments, kits contain components in pre-measured unit amounts to minimize the need for measurements by end-users. In some embodiments, kits include instructions for performing one or more of the disclosed methods. Preferably, the kit components are optimized to operate in conjunction with one another.

In some embodiments, the present teachings comprise a kit for detecting the formation of an siRNA-containing RISC, comprising; a modified siRNA construct, wherein the modified siRNA construct comprises an anti-sense strand and a sense strand, wherein the anti-sense strand comprises a 3′ end, wherein the 3′ end comprises a fluorophore, and wherein the sense strand comprises a 5′ end, wherein the 5′ end comprises a quencher; and, transfection reagents.

In some embodiments, the present teachings provide a kit for detecting the formation of an siRNA-containing RISC, comprising; a modified siRNA construct, wherein the modified siRNA construct comprises an anti-sense strand and a sense strand, wherein the anti-sense strand comprises a 3′ end, wherein the 3′ end comprises a quencher, and wherein the sense strand comprises a 5′ end, wherein the 5′ end comprises a fluorophore; and, transfection reagents.

In some embodiments, the transfection reagents are selected from the group consisting of CaCl2, or liposomes. In some embodiments, the kits can further contain reagents for performing an in situ hybridization.

In some embodiments, the present teachings also provide compositions, which may be included in the kits. Thus, in some embodiments, the present teachings provide a composition comprising; a modified siRNA construct, wherein the modified siRNA construct comprises an anti-sense strand and a sense strand, wherein the anti-sense strand comprises a 3′ end, wherein the 3′ end comprises a fluorophore, and wherein the sense strand comprises a 5′ end, wherein the 5′ end comprises a quencher. In some embodiments, the present teachings provide a composition comprising; a modified siRNA construct, wherein the modified siRNA construct comprises an anti-sense strand and a sense strand, wherein the anti-sense strand comprises a 3′ end, wherein the 3′ end comprises a quencher, and wherein the sense strand comprises a 5′ end, wherein the 5′ end comprises a fluorophore.

In some embodiments, the 3′ end of the sense strand further comprises a cell uptake element. In some embodiments, the cell uptake element is an HIV tat transduction domain. In some embodiments, the cell uptake element is a G-PNA.

### Example

Transfection of cells of interest is performed with a modified siRNA construct as depicted in FIG. 2. The transfection can employ the Silencer® CellReady™ Transfection kit from Ambion. Briefly, adherent cells are trypsinized, and resuspended in normal growth medium at the desired concentration. Cells are then set aside at 37 C while the modified siRNA construct and transfection agent is prepared. The modified siRNA construct is mixed with the Opti-MEM1 and incubated for 10 minutes, along with appropriate controls reactions. The transfection agent is then diluted into an Optimization Plate, and the plate is rocked gently back and forth to evenly distribute the complexes and to resuspend the siRNA. The plate is then incubated for ten minutes at room temperature. Cells are then transferred to the Optimization Plate. The cells are then mixed with the modified siRNA construct/transfection agent. After a twenty-four incubation, the transfection media is replaced with fresh normal growth media. Fluorescent measurements can then be taken, resulting for example in a graph as depicted in FIG. 1.

The anti-sense strand sequence and sense strand sequence for such an experiment as depicted in FIG. 2 for GAPDH are, respectively:

| | |
|---|---|
| SEQ ID NO:1 | 5′AAAGUUGUCAUGGAUGACCTT3′ |
| SEQ ID NO:2 | 5′GGUCAUCCAUGACAACUUUTT3′ |

Although the disclosed teachings have been described with reference to various applications, methods, and kits, it will be appreciated that various changes and modifications may be made without departing from the teachings herein. The foregoing examples are provided to better illustrate the present teachings and are not intended to limit the scope of the teachings herein. Certain aspects of the present teachings may be further understood in light of the following claims.

We claim:

1. A method for detecting the formation of an siRNA-containing RISC comprising;
providing a modified siRNA construct, wherein the modified siRNA construct comprises an anti-sense strand and a sense strand, wherein the anti-sense strand comprises a 3′ end, wherein the 3′ end comprises a fluorophore, and wherein the sense strand comprises a 5′ end, wherein the 5′ end comprises a quencher;
transfecting the modified siRNA construct into a sample; and,
measuring fluorescence to detect the formation of the siRNA-containing RISC.

2. The method according to claim 1 wherein the 3′ end of the sense strand further comprises a cell uptake element.

3. The method according to claim 2 wherein the cell uptake element is an HIV tat transduction domain.

4. The method according to claim 2 wherein the cell uptake element is a G-PNA.

5. A method for detecting the formation of an siRNA-containing RISC comprising;
providing a modified siRNA construct, wherein the modified siRNA construct comprises an anti-sense strand and a sense strand, wherein the anti-sense strand comprises a 3′ end, wherein the 3′ end comprises a quencher, and wherein the sense strand comprises a 5′ end, wherein the 5′ end comprises a fluorophore;
transfecting the modified siRNA construct into a sample; and,
measuring fluorescence to detect the formation of the siRNA-containing RISC.

6. The method according to claim 5 wherein the 3′ end of the sense strand further comprises a cell uptake element.

7. The method according to claim 6 wherein the cell uptake element is an HIV tat transduction domain.

8. The method according to claim 6 wherein the cell uptake element is a G-PNA.

9. A composition comprising;
a modified siRNA construct, wherein the modified siRNA construct comprises an anti-sense strand and a sense strand, wherein the anti-sense strand comprises a 3′ end, wherein the 3′ end comprises a fluorophore, and wherein the sense strand comprises a 5′ end, wherein the 5′ end comprises a quencher.

10. The composition according to claim 9 wherein the 3′ end of the sense strand further comprises a cell uptake element.

11. The composition according to claim 10 wherein the cell uptake element is an HIV tat transduction domain.

12. The composition according to claim 10 wherein the cell uptake element is a G-PNA.

13. A composition comprising;
a modified siRNA construct, wherein the modified siRNA construct comprises an anti-sense strand and a sense strand, wherein the anti-sense strand comprises a 3′ end, wherein the 3′ end comprises a quencher, and wherein the sense strand comprises a 5′ end, wherein the 5′ end comprises a fluorophore.

14. The composition according to claim 13 wherein the 3′ end of the sense strand further comprises a cell uptake element.

15. The composition according to claim 14 wherein the cell uptake element is an HIV tat transduction domain.

16. The composition according to claim 14 wherein the cell uptake element is a G-PNA.

17. A composition comprising a RISC and an anti-sense strand of a modified siRNA construct, wherein the anti-sense strand comprises a 3′ end, wherein the 3′ end comprises a fluorophore.

18. A composition comprising a RISC and an anti-sense strand of a modified siRNA construct, wherein the anti-sense strand comprises a 3′ end, wherein the 3′ end comprises a quencher.

19. A kit for detecting the formation of an siRNA-containing RISC, comprising;
a modified siRNA construct, wherein the modified siRNA construct comprises an anti-sense strand and a sense strand, wherein the anti-sense strand comprises a 3′ end, wherein the 3′ end comprises a fluorophore, and wherein the sense strand comprises a 5′ end, wherein the 5′ end comprises a quencher; and,
transfection reagents.

20. A kit for detecting the formation of an siRNA-containing RISC, comprising;
a modified siRNA construct, wherein the modified siRNA construct comprises an anti-sense strand and a sense strand, wherein the anti-sense strand comprises a 3′ end, wherein the 3′ end comprises a quencher, and wherein the sense strand comprises a 5′ end, wherein the 5′ end comprises a fluorophore; and,
transfection reagents.

21. The kit according to claim 19 or 20, wherein the transfection reagents are selected from the group consisting of CaCl₂, or liposomes.
